SEQUENCE LISTING (9 sequences)

SEQ ID NO: 1 - protein, 157 aa, Homo sapiens [sequence omitted]

SEQ ID NO: 2 - protein, 26 aa, Artificial Sequence
  Fusion of two homo sapiens TNF alpha peptides in branching structure with T helper sequence from Tetanus toxid, linkers and lipid cap
RTPSDKPVAHKPWYEPIYLGGVFQLE

SEQ ID NO: 3 - protein, 24 aa, Artificial Sequence
  Fusion of a T helper sequence with a polar sequence, spacers with attachment to the lipid, dipalmitoyl-S-glyceryl on the N-terminal Cys
CSKKKKSSQYIKANSKFIGITELS

SEQ ID NO: 4 - protein, 20 aa, homo sapiens
CSRTPSDKPVAHVVANPQAE

SEQ ID NO: 5 - protein, 41 aa, Artificial Sequence
  Sequence combining homo sapiens TNF epitope A2 and F with linkers
CSRTPSDKPVAHVVANPQAEGPSLKPWYEPIYLGGVGQLEK

SEQ ID NO: 6 - protein, 43 aa, Artificial Sequence
  Peptide combining linkers T helper pepide and homo sapiens TNF epitope F
CSKKKKSQYIKANSKFIGITELGPSLKPWYEPIYLGGVGQLEK

SEQ ID NO: 7 - protein, 45 aa, Artificial Sequence
  Fused seqeunce of linkers T helper sequence polar sequence and homosapiens TNF A2 epitope
CSKKKKSQYIKANSKFIGITELGPSLSRTPSDKPVAHVVANPQAE

SEQ ID NO: 8 - protein, 66 aa, Artificial Sequence
  Fused sequence of linkers, T helper sequence and both homo sapiens epitopes A2 and F
CSKKKKSQYIKANSKFIGITELGPSLSRTPSDKPVAHVVANPQAEGPSLKPWYEPIYLGGVGQLEK

SEQ ID NO: 9 - protein, 18 aa, homo sapiens
CKPWYEPIYLGGVFQLEK

(19) United States
(12) Patent Application Publication
(10) Pub. No.: US 2015/0225478 A1
(43) Pub. Date: Aug. 13, 2015

(54) Immunogenic and Prophylactic Compositions, Methods of Making Same, and Methods for Treating and Preventing TNF-Mediated Disease

(71) Applicant: Thymon, LLC, New York, NY (US)

(72) Inventor: Gideon Goldstein, New York, NY (US)

(21) Appl. No.: 14/695,807

(22) Filed: Apr. 24, 2015

Related U.S. Application Data
(63) Continuation of application No. PCT/US2014/012678, filed on Jan. 23, 2014.
(60) Provisional application No. 61/768,055, filed on Feb. 22, 2013.
(60) Provisional application No. 61/756,583, filed on Jan. 25, 2013.

(51) Int. Cl. C07K 16/24

(52) U.S. Cl. CPC C07K 16/241; C07K 2317/34; C07K 2317/76

(57) ABSTRACT

An immunogenic composition comprises a peptide that induces in vivo TNF monomer-specific antibodies that bind an epitope of dissociated monomers of human TNF, the epitope being located wholly or partially within a TNF monomer-monomer interface region. Antibodies induced in vivo by this composition disrupt assembly or prevent re-assembly of the TNF monomers into bioactive trimeric human sTNF. The composition can comprise an additional peptide that induces in vivo TNF monomer-specific antibodies that bind another epitope of dissociated monomers of human TNF. Methods of making these immunogenic compositions are also described. Methods for treating a subject having a disease mediated by human TNF comprise administering a described immunogenic compositions which induces antibodies in a treated subject that disrupt or reduce the in vivo assembly or reassembly of dissociated monomers of TNF into bioactive trimeric human sTNF. These compositions and methods do not affect the structure or bioactivity of tmTNF or increase the treated subject's susceptibility to infection by an intracellular pathogen.

### CROSS-REFERENCE TO RELATED APPLICATIONS

This application is a continuation of international patent application No. PCT/2014/12678, filed on Jan. 23, 2014, which claims priority of U.S. provisional patent application No. 61/768,055, filed Feb. 22, 2013 (now expired) and U.S. provisional patent application No. 61/756,583, filed Jan. 25, 2013 (now expired). All patent applications are incorporated herein by reference.

### INCORPORATION-BY REFERENCE OF MATERIAL SUBMITTED IN ELECTRONIC FORM

Applicant hereby incorporates by reference the Sequence Listing material filed in electronic form herewith. This file is labeled GGP14C1USA_ST25.txt″, was created on Apr. 21, 2015, and is 7 KB in size.

### BACKGROUND OF THE INVENTION

Tumor necrosis factor (TNF; previously referred to as tumor necrosis factor-α) is a proinflamatory cytokine that plays a major role in the pathogenesis of rheumatoid arthritis and associated inflammatory diseases, such as ankylosing spondylitis, juvenile rheumatoid arthritis, and psoriatic arthritis. The human proform, transmembrane-bound TNF (tmTNF), is a 26-kDa homotrimer comprising three non-covalently associated monomers, each monomer having N-terminal sequence imbedded in the cell membrane. Each monomer of tmTNF has a 233 amino acid sequence (UniProtKB/Swiss-Prot entry Accession No. P01375). Soluble TNF (sTNF) is a homotrimer formed by enzymatic cleavage from its pro-form tmTNF. Each monomer of the sTNF trimer has a 157 amino acid sequence (SEQ ID NO: 1), which is the same sequence as aa77 to 233 of the published Acc No. P01374.

Both forms of active TNF (tmTNF and sTNF) exist as homotrimers¹⁰⁻¹³ and engage trimeric receptors that recognize receptor-binding sites in the grooves between the TNF monomers in assembled homotrimers. The grooves between the monomers comprise amino acid sequence from two contiguous monomers^{14,15}. The receptor binding regions of both forms of TNF are identical.

Trimer integrity is essential for biological function. For tmTNF, trimeric structure is established intracellularly before tmTNF insertion into the cell membrane and is maintained in tmTNF by the anchoring of the protein stems passing through the membrane plus further lipid anchoring by palmitoylated amino acid side chains at the membrane boundary¹⁷. In contrast, sTNF active trimers dissociate freely into inactive monomers SEQ ID NO: 1 and dimers that reform as active sTNF homotrimers in a steady-state equilibrium between the three forms¹⁸.

Anti-TNF biologics have provided a major advance in the management of the above-noted inflammatory diseases with anti-TNF monoclonal antibodies REMICADE (Infliximab; Janssen Biotech, Inc.) and HUMIRA (Adalimumab, Abbott Laboratories), and a chimeric solubilized TNF receptor fused to Fc, i.e., ENBREL (Entanercept, Biogen, Inc) being widely used^{1,2}. This therapeutic and marketing success is marred by the rare but statistically significant occurrence of serious infections and malignancies^{3,4}, likely related to concomitant blockade of tmTNF^{5,6} function impairing immune defenses. These adverse occurrences have included the development of tuberculosis, systemic fungal infection and other intracellular infections due principally to intracellular pathogens such as Mycobacterium tuberculosis, Listeria monocytogenes and Histoplasma capsulatum, and certain forms of cancer. These results were unsurprising since, while these therapeutic agents block pro-inflammatory sTNF, they also block tmTNF which is essential for juxtacrine cellular control of such intracellular infections and malignancies^{3,7,9}.

Because the receptor binding regions of both forms of TNF are identical, there has been little hope for the development of new therapeutic agents that selectively block receptor engagements of one form versus the other. Antibodies to short sequences of TNF have not lead to useful therapeutics. For example, in 1987, Socher et al.²⁶ in exploring antibodies to full or partial synthetic sequences of TNF, observed a high polyclonal antibody response to the TNF fragment 1-15 that appeared to block bioactivity and receptor binding of TNF. However, this 16-year old observation has not lead to the development of additional therapeutic reagents, likely because the TNF receptor is a discontinuous surface region not associated with TNF amino acids 1-15. Subsequent researchers in 2001 coupled TNF amino acids 4-23 to papillomavirus-like particles, and observed an induction of polyclonal antibodies, and an attenuation of experimental arthritis²⁷. Other researchers in 2007 used the same fragment TNF aa4-23 coupled to a virus-like particle-based composition and induced antibodies that attenuated experimental arthritis. No suppression of resistance to infection occurred, in contrast with full length TNF immunization²⁸. Because these TNF fragments were not directed to receptor binding regions of TNF, these publications displayed no further teachings or suggestion of therapeutic use of the resulting polyclonal antibodies; and further research has not been published since that date.

One more recent attempt to selectively suppress the pro-inflammatory activity of sTNFα while preserving tmTNFα function required for innate immunity involved the design of synthetic dominant-negative TNFα monomer variants that formed trimers that were inactive¹⁹. These were shown to attenuate experimental arthritis without suppressing innate immunity to infection²⁰, emphasizing the major role of sTNFα in pathogenesis of arthritis. Another approach has been the search for small-molecule drugs that interact with the inter-monomer contact regions. One molecule, SP304, bound such a contact region with μM affinity to effect trimer disruption in vitro^{21,22}.

Despite the plethora of literature in the field of anti-TNFα treatment for a variety of inflammatory disorders, there remains a need in the art for new and useful compositions and methods for generating therapeutic or prophylactic immunogenic compositions for these diseases which do not result in adverse side effects due to suppression of cellular immunity.

### SUMMARY OF THE INVENTION

As described herein the inventor has provided selective anti-TNF monomer-specific biologic compositions and various methods of use thereof which do not affect the structure or bioactivity of tmTNF or increase the treated subject's susceptibility to infection by an intracellular pathogen.

In one aspect, an immunogenic composition comprises a peptide that induces in vivo TNF monomer-specific antibodies that bind an epitope of dissociated monomers of human TNF. Each epitope is located wholly or partially within a TNF monomer-monomer interface region. In one embodiment, a TNF monomer-specific epitope is epitope A2, i.e., PSDKPVAH or PSDKVPAHV, amino acids 8-15 or 8-16 of SEQ ID NO: 1. In another embodiment, a TNF monomer-specific epitope is epitope F, EPIYLGGVF, amino acids 116-124 of SEQ ID NO: 1. In another embodiment, the TNF monomer-specific epitope is epitope A1, SSRTPSKDP, amino acids 4-12 of SEQ ID NO: 1. Antibodies induced in vivo by certain of these compositions, e.g., those that contain a peptide that induces antibodies to monomer-specific epitope A2 or F, disrupt assembly of the monomers into bioactive trimeric human sTNF.

In another aspect, the composition described herein further comprises an additional peptide that induces in vivo TNF monomer-specific antibodies that bind an additional epitope of dissociated monomers of human sTNF. A different epitope is also located wholly or partially within a TNF monomer-monomer interface region. Antibodies induced to this composition disrupt assembly of the monomers into bioactive trimeric human sTNF in vivo. In one embodiment, a TNF monomer-specific epitope is epitope A2, i.e., PSDKPVAH or PSDKVPAHV, amino acids 8-15 or 8-16 of SEQ ID NO: 1. In another embodiment, the TNF monomer-specific epitope is epitope F, EPIYLGGVF, amino acids 116-124 of SEQ ID NO: 1. In still another embodiment, the immunogenic composition comprises both an immunogen that induces anti-A2 monomer-specific antibodies and an immunogen that induces anti-F monomer-specific antibodies, presented in separate constructs or in a single construct as described below. In another aspect, the immunogenic composition presents the peptide in various constructs, e.g., within a virus-like particle, in a liposome or as a self-adjuvanting synthetic construct.

In still another aspect, the compositions described herein are provided for use in the treatment of a disease mediated by the activity of sTNF or preventing or retarding such disease, as described in detail below. In one embodiment, the disease is rheumatoid arthritis (RA), juvenile rheumatoid arthritis, ankylosing spondylitis (AS), psoriatic arthritis or psoriasis.

In still other aspects, methods of producing such immunogenic compositions and combining them with suitable pharmaceutical carriers, adjuvants, as needed, or excipients is described.

In still another aspect, a method for treating a subject having a disease mediated by the activity of TNF or preventing or retarding such disease involves inducing in vivo in a mammalian subject anti-TNF monomer-specific antibodies that bind an epitope of dissociated monomers of human TNF. The epitope is located wholly or partially within a TNF monomer-monomer interface region, and the binding disrupts assembly of the monomers into bioactive trimeric human sTNF. In one embodiment, the method involves administering to a mammalian subject in need thereof an effective antibody-inducing amount of a composition as described herein.

In still another aspect, a method for treating a subject having a disease mediated by human TNF comprises reducing the amount, concentration or bioactivity of sTNF in the blood of a subject having the disease without affecting the amount, concentration or bioactivity of tmTNF. This is accomplished by disrupting, preventing or reducing the in vivo assembly or reassembly of dissociated monomers of TNF into bioactive trimeric human sTNF without affecting the amount, concentration or bioactivity of transmembrane TNF. In certain embodiments, this method employs the compositions described above and herein. In one embodiment, the disease is rheumatoid arthritis (RA), juvenile rheumatoid arthritis, ankylosing spondylitis (AS), psoriatic arthritis or psoriasis.

In still another aspect, sTNF elevations are also implicated in HIV infection and type II diabetes.

In still another aspect, the immunogenic composition described above is useful in a method of retarding, inhibiting or preventing initial HIV-1 infection in a mammalian subject. Such a method involves administering to a subject in need thereof a composition that induces TNF monomer-specific antibodies that disrupt assembly of the TNF monomers into bioactive trimeric human sTNF, optionally in combination with known HIV therapies.

In yet another aspect, a method for treating a subject with type II diabetes comprises administering periodically to a subject in need thereof an isolated or synthetic selective anti-TNF monomer-specific immunogenic composition, or pharmaceutical composition, as described above, optionally in combination with known anti-diabetic therapies.

In yet another aspect, a method of making the compositions described herein involves synthesizing one or more of the TNF monomer-specific epitope peptides or immunogen into an amino acid construct, optionally containing flanking amino acids for coupling to a T helper epitope or other carrier. In one embodiment, these constructs are prepared as fully-synthetic self-adjuvanting vaccines.

Other aspects and advantages of these methods and compositions are described further in the following detailed description.

### BRIEF DESCRIPTION OF THE DRAWINGS

FIG. 1 illustrates the immunogen and epitope detector sequences for TNF interface A and TNF Interface F, which were used to identify the sequences and margins of the three TNF epitopes A1, A2 and F described herein as well as the binding activities of the anti-TNF antibodies generated thereto. These sequences are discussed in Example 1 below.

FIG. 2A is a bar graph illustrating the monomer-specific TNF epitopes by immunizing a rat with TNF amino acids 1-23. Antibody binding responses were measured on recombinant TNF (rTNF), a mixture of monomers, dimers and trimers, and on synthetic peptides employing the indicated terminally truncated TNF peptides. The synthetic detector peptides included: TNF amino acids 1-10 of SEQ ID NO: 1, which demonstrated no binding at all; TNF amino acids 5-12 of SEQ ID NO: 1, minimal to no binding; the A1 epitope SSRTPSDKP, TNF amino acids 4-12 of SEQ ID NO: 1; TNF amino acids 4-11 of SEQ ID NO: 1 SSRTPSDK; TNF amino acids 9-15 of SEQ ID NO: 1 SDKPVAH; the A2 epitope sequence of amino acids 8-15 of SEQ ID NO: 1; TNF amino acids 8-14 of SEQ ID NO: 1 PSDKPVA; and TNF amino acids 16-23 of SEQ ID NO: 1 VVANPQAE, which exhibited no binding. Clearly only two overlapping epitopes were detected by rat antiserum to TNF amino acids 1-23, which were epitope A1, TNF amino acids 4-12 and A2, TNF amino acids 8-15.

FIG. 2B is a bar graph illustrating the margins of the A2 epitope PSDKPVAHV detected in mouse serum using the same procedure described in FIG. 2A, with synthetic peptides. The synthetic detector peptides included: TNF amino acids 10-17 of SEQ ID NO: 1 DKPVAHVV, which demonstrated minimal binding; TNF amino acids 9-16, minimal binding; the A2 epitope PSDKPVAHV, TNF amino acids 8-16 of SEQ ID NO: 1; TNF amino acids 8-15 of SEQ ID NO: 1 PSDKPVAH; and TNF amino acids 8-14 of SEQ ID NO: 1 PSDKPVA, no binding. When rat and rabbit sera were used, the boundaries of epitope A2 are amino acids 8-15 of SEQ ID NO: 1. The immune system of the mouse sees only TNF amino acids 8-16 and does not bind to TNF amino acids 8-15, as shown in the graph.

FIG. 2C is a bar graph illustrating the results of determining the margins of the monomer-specific TNF epitopes by immunizing a rat or mouse with TNF amino acids 112-128 of SEQ ID NO: 1, KPWYEPIYLGGVFQLEK (interface region underlined; F epitope in bold). Antibody binding responses were measured on recombinant TNF (rTNF), a mixture of monomers, dimers and trimers, and on synthetic peptides employing the indicated four terminally truncated TNF peptides. The synthetic peptides were IYLGGVF, amino acids 118-124 of SEQ ID NO: 1; PIYLGGVF, amino acids 117-124 of SEQ ID NO: 1, EPIYLGGVF, amino acids 116-124 of SEQ ID NO: 1 (epitope F) and EPIYLGGV, amino acids 116-123 of SEQ ID NO: 1. The greatest binding was to the aa116-124 peptide, thereby indicating the margins of the epitope (referred to as epitope F).

FIG. 3A illustrates data from the sandwich assay using 200 ng/mL sTNF, biotin-labeled antibody and non-biotin labeled antibody, as described in Example 2 below. The binding curves show that commercial REMICADE anti-TNF antibody, when labeled with biotin and mixed with TNF (trimers, dimers and monomers), binds multimeric forms of TNF. The biotinylated antibody-TNF in the mixture still has available TNF trimer epitopes that can bind and form a sandwich with the unlabeled plated REMICADE antibody (•). In contrast, Protein A/Protein G purified IgG (∘) was obtained from rats immunized with TNF amino acids 1-23 of SEQ ID NO: 1. This purified IgG contains a mixture of monomer-specific anti-TNF that selectively bind epitope A1 and monomer-specific anti-TNF that selectively bind epitope A2. The purified IgG (∘) does not sandwich in the assay, because once these antibodies bind the TNF monomers in the TNF mixture, the labeled monomer-specific antibody-TNF complexes have no available monomer-specific epitopes to bind to the plated unlabeled monomer-specific antibody on the plate. Labeled monomer-specific antibody-TNF monomers complexes are simply washed from the plate without binding. REMICADE antibody that binds trimeric TNF was used as a positive control in this assay. Thus neither anti-A1 nor anti-A2 antibodies bind the trimeric form of TNF.

FIG. 3B illustrates data from a similar sandwich assay to that of FIG. 3A, using as reagents: commercial REMICADE anti-TNF antibody (•); affinity purified IgG from antisera to the TNF epitope F that selectively binds only epitope F: EPIYLGGVF (X) and Protein A/G purified IgG from antisera to the TNF epitope F that selectively binds only epitope F: EPIYLGGVF (□). Thus, in contrast to the commercial REMICADE anti-TNF antibodies, the anti-F antibodies do not bind the trimeric form of TNF.

FIG. 4A illustrates the results of an assay of antibody inhibition of sTNF-induced cytotoxicity in target cells, using antiserum generated to TNF amino acids 1-23 that contains antibodies that selectively bind the epitopes PSDKPVAH and SSRTPSDKP (epitopes A2 and A1, respectively). The titers of antisera with 200 pg/mL TNF in all wells are displayed under the bars, from 1×10⁶, 2.5×10⁵, 1×10⁵, 2.5×10⁴, 1×10⁴, and 2.5×10³. The last bar is sTNF in 50% NRS.

FIG. 4B illustrates functional blocking of sTNF cytotoxicity in actinomycin-treated WEHI cells in the assay described in Example 2 by antiserum to TNF epitope F. The indicated dilutions (50%, 16.7%, 5.6%, 1.85%, 0.48%) of monomer-specific antisera generated to the immunogen KPWYEPIYLGGVFQLEK, amino acids 112-128 of SEQ ID NO: 1 (the F beta sheet interface sequence of TNF), in rats were compared for their ability to inhibit sTNF bioactivity with 200 pg/mL TNF and 50% NRS (normal rat serum). As is shown in this figure, inhibition of TNF cytotoxicity was shown using antiserum diluted from 0.48 to 50%. Thus, the monomer-specific antisera to epitope F showed the ability to inhibit the cytotoxic effect of sTNF on the cells as evidenced by increasing replication of cells in the presence of the antisera. Statistical significance was determined by one-way ANOVA and post testing with Dunnett's test. REMICADE antibody, which binds trimeric TNF, was used as a positive control in this assay.

FIG. 5 is a bar graph showing inhibition of sTNF cytotoxicity in WEHI cells by monoclonal antibody generated to TNF epitopes A1 or A2. Cell replication (OD) was measured in WEHI cells grown in the presence of no TNF and the cells showed good replication. WEHI cells grown in the presence of 0.2 ng/ml full length TNF 1-157 of SEQ ID NO: 1 demonstrated that TNF inhibited replication. Cells grown in TNF plus the commercial REMICADE anti-TNF antibody at 1 μg/mL, showed that the antibody returned replication to the same levels demonstrated in the absence of TNF. TNF plus the inventor's monoclonal antibody A1-4H6 to TNF epitope A1 (amino acids 4-12 of SEQ ID NO: 1) at 10 μg/mL, failed to inhibit WEHI cell replication, which remains at the levels of cells exposed to TNF alone. WEHI cells were cultured in the presence of TNF plus the inventor's monoclonal antibody A2-8D12 to TNF epitope A2 (amino acids 8-16 of SEQ ID NO: 1) at 10 μg/mL; and TNF plus the inventor's monoclonal antibody A2-10H10 to TNF epitope A2 (amino acids 8-16 of SEQ ID NO: 1) at 0.25 μg/mL. Data from these two latter monoclonal antibodies showed highly statistically significant inhibition of sTNF cytotoxicity (i.e., reduction of cell killing). The second monoclonal antibody A2-10H10 showed high sensitivity, a 40× increase in potency in this assay over the other anti-A2 antibody A2-8D12. These results demonstrate that anti-A2 monoclonal antibodies inhibit sTNF and that one such antibody A2-10H10 exhibits a higher affinity than the other, as demonstrated by the 40 fold lower dose. No inhibition even at a high dose was demonstrated by the anti-A1 monoclonal antibody. These data demonstrate that the effects of the TNF amino acid 1-23 polyclonal antisera were due to the anti-A2 antibodies only.

### DETAILED DESCRIPTION OF THE INVENTION

The inventor has provided selective anti-TNF monomer-specific biologic compositions and various methods of use based on the determination that the induction of antibodies directed to selected epitopes partially or fully within the internal interface contact regions of TNF free monomers will block their associations with other monomers and cause progressive disruption of bioactive sTNF trimer formation. It is advantageous to have an immunogenic composition that induces in vivo an antibody that selectively blocks the activity of sTNF but not tmTNF for the treatment of rheumatoid arthritis (RA), juvenile rheumatoid arthritis, ankylosing spondylitis (AS), psoriatic arthritis (PA), and other chronic inflammatory diseases, among other diseases.

The Immunogenic Compositions

The inventor determined that antibodies directed to an epitope partially or fully within the internal interface contact region of free TNF monomers would block their associations with other monomers and cause progressive disruption of trimer formation.

In contrast to the known publications on TNF, the inventor determined that there were two overlapping epitopes in the TNF sequence of amino acids 1-15 of SEQ ID NO: 1. One epitope A1 which spanned amino acids 4-12 of SEQ ID NO: 1 was monomer-specific, but did not disrupt trimer formation. The other epitope A2 spanned amino acids 8-15 or 8-16 of SEQ ID NO: 1, was monomer-specific and did disrupt trimer formation. Further the inventor identified a new unrecognized epitope F, amino acids 116-124 of SEQ ID NO: 1 in the F sheet of TNF, which was monomer-specific and did disrupt trimer formation. The discovery of, and methods for use of, immunogenic constructions that induce antibodies that specifically bind these epitopes is discussed in detail below and in the examples.

Early x-ray crystallography studies established that the 157 amino acid (SEQ ID NO: 1) TNF monomers formed an “elongated, anti-parallel β pleated sheet sandwich with “jelly-roll” topology”. Three monomers in intimate but non-covalent association constituted the active sTNF trimer¹¹. Five stretches of amino acid sequences formed the interface β sheet contact surfaces: A, aa11-18; A′, aa35-39; C, aa54-67; F, aa114-126 and H, aa149-157, all of SEQ ID NO: 1, where A, A′, C, F and H refer to a β sheet naming convention¹¹. The inventor explored all five regions for potential B cell epitopes and detected and mapped antibodies to two epitopes partially (the A β sheet) and one epitope fully (the F β sheet) within an interface region, all being outside the known regions of the receptor binding sites of TNF^{12,13,15}. See, Example 1 below.

Thus, in one embodiment, an immunogenic composition comprises a peptide that induces in vivo TNF monomer-specific antibodies that bind an epitope of dissociated monomers of human TNF SEQ ID NO: 1, the epitope being located wholly or partially within a TNF monomer-monomer interface region. The binding disrupts or prevents assembly of the monomers into bioactive trimeric human sTNF.

In another embodiment, the immunogenic composition includes an additional or different peptide that induces in vivo TNF monomer-specific antibodies that bind an additional or different epitope of dissociated monomers of human TNF. In the same manner as the first immunogen, the epitope is located wholly or partially within a TNF monomer-monomer interface region, and the binding disrupts in vivo assembly of the monomers into bioactive trimeric human sTNF. It is also contemplated that an immunogenic composition contains two or more such TNF monomer-specific antibody-inducing peptides that are selective for different epitopes.

In one embodiment, the immunogenic composition is designed to actively induce antibodies against the TNF monomer-specific A2 epitope having the sequence PSDKPVAH, amino acids 8-15 of SEQ ID NO: 1 or sequence PSDKPVAHV, amino acids 8-16 of SEQ ID NO: 1. In another embodiment, the immunogenic composition is designed to actively induce antibodies against the TNF monomer-specific A1 epitope having the sequence SSRTPSDKP, amino acids 4-12 of SEQ ID NO: 1. In yet another embodiment, the immunogenic composition is designed to actively induce antibodies against the TNF monomer specific F epitope having the sequence EPIYLGGVF, amino acids 116 to 124 of SEQ ID NO: 1. As described below, antibodies to the A1 epitope, while TNF monomer-specific, have been found to be inactive in blocking TNF function when used alone. Antibodies to the A2 and F epitopes specifically bind TNF monomers but not trimers, disrupt assembly of sTNF trimers and inhibit sTNF function in vitro. These monomer-specific anti-A2 and anti-F epitope antibodies do not bind transmembrane TNF (tmTNF) and do not affect the structure or bioactivity of tmTNF. Additionally, these antibodies do not bind intact bioactive trimeric human sTNF. In still other embodiments, the immunogenic composition is designed to actively induce antibodies against the TNF monomer-specific A2 epitopes PSDKPVAH or PSDKPVAHV and the F epitope EPIYLGGVF.

A variety of immunogenic peptides for use in the immunogenic compositions that induce selective antibodies to a selective monomer-specific TNF epitope above can be readily generated and provided in a variety of forms. In one embodiment, the TNF monomer-specific antibody-inducing peptide is simply the minimum epitope sequence or sequences for a single epitope as defined herein. In other embodiments, the immunogenic peptide may contain the epitope sequence flanked by other amino acids, so that the immunogenic antibody-inducing peptide sequence is between 8 and about 30 amino acids in length, including peptides of 8, 9, 10, 11, 12, 13, 14, 15, 16, 17, 18, 19, 20, 21, 22, 23, 24 or 25 amino acids in length. The identity of the flanking amino acids is not essential to the biological function of the immunogenic peptide. In one embodiment, the flanking amino acids in the antibody-inducing peptides are for convenience the amino acids that normally flank the epitope in the naturally occurring TNF sequence. For example, one such useful immunogenic peptide is SRTPSDKPVAH, amino acids 5-15 of SEQ ID NO: 1 or a fragment thereof. In another embodiment, the TNF monomer-specific antibody-inducing peptide is C-SRTPSDKPVAHVVANPQAE SEQ ID NO: 4. In another embodiment, a useful TNF monomer-specific antibody-inducing peptide is KPWYEPIYLGGVFQLEK, amino acids 112-128 of SEQ ID NO: 1, or a fragment thereof. Still other peptides comprising the minimum epitopes with desired flanking amino acids can be generated from the known sequence of TNF, using the information provided above. In certain embodiments, the peptide is optionally amidated on the carboxy terminus. In other embodiments, the peptide contains an amino acid, such as a Cys on the N-terminus for attachment to a carrier. Among such carriers include a tetanus toxoid or diphtheria toxoid. The flanking amino acid may also be attached to another sequence such as a T helper sequence.

The production of immunogenic compositions that induce antibodies that specifically bind to one of epitopes, A2: PSDKPVAH or PSDKPVAHV, or F: EPIYLGGVF can employ conventional techniques. In some embodiments, a single TNF monomer-specific antibody-inducing peptide is formulated in a pharmaceutical composition. Alternatively, two or more different such peptides may be prepared similarly and combined in a single immunogenic composition. In still another embodiment, the immunogenic peptide may be coupled to a carrier protein. Alternatively two or more such constructs may be mixed together to form a single composition.

In one embodiment, a TNF monomer-specific antibody-inducing peptide may be made synthetically by conventional methods of chemical synthesis or recombinantly by expression in a selected host cell, also by now-conventional means. Optional amino acids (e.g., -Gly-Ser-) or other amino acid or chemical compound spacers may be included at the termini of the peptides for the purpose of linking the peptides together or to a carrier. In one embodiment, the immunogenic composition may take the form of one or more of the above-described peptides expressed as a synthetic peptide coupled to a carrier protein. In another embodiment, the peptide or peptides is/are expressed within a recombinantly produced protein, optionally fused in frame with a carrier protein.

In still another aspect, the selected peptides may be linked sequentially and produced synthetically or expressed within a recombinantly produced protein, with and without spacer amino acids there between, to form a larger recombinant peptide or protein. Alternatively, the recombinant protein may be fused in frame with a carrier protein.

In another embodiment, a single immunogenic peptide can be generated to induce antibodies to more than a single epitope. For example, one peptide is capable of inducing antibodies that bind both epitope A2 and epitope F. It is anticipated that such multi-specific immunogenic constructs that induce, e.g., the antibodies reactive with A2 and F, will enhance avidity and create greater potency than the single antibody-inducing immunogenic peptides alone. In one embodiment, for example, a single immunogenic peptide that induces antibody to TNF monomer specific epitope A2 and epitope F has the sequence CSRTPSDKPVAHVVANPQAEGPSLKPWYEPIYLGGVFQLEK SEQ ID NO: 5. This peptide, in which the two epitopes are bolded and a linker is italicized, is optionally amidated on the carboxy terminus. It is well within the skill of the art given the teachings herein to create other immunogenic peptides comprising the A2 and F epitopes. In one embodiment, for example the epitope order may change; or the linker or flanking sequences may changes.

For embodiments which are associated with a carrier protein, e.g., through the Cys on the N terminus, the carrier protein can be a protein or other molecule which can enhance the immunogenicity of the selected immunogen. Such a carrier may be a larger molecule which has an adjuvanting effect. Exemplary conventional protein carriers include, without limitation, E. coli DnaK protein, galactokinase (galK, which catalyzes the first step of galactose metabolism in bacteria), ubiquitin, α-mating factor, β-galactosidase, and influenza NS-1 protein. Toxoids (i.e., the sequence which encodes the naturally occurring toxin, with sufficient modifications to eliminate its toxic activity) such as diphtheria toxoid and tetanus toxoid may also be employed as carriers. Similarly a variety of bacterial heat shock proteins, e.g., mycobacterial hsp-70 may be used. Glutathione reductase (GST) is another useful carrier. One of skill in the art can readily select an appropriate carrier.

In yet another embodiment, the peptide immunogens may be in the form of a multiple antigenic peptide (“MAP”, also referred to as an octameric lysine core peptide) construct. An immunogenic composition may contain multiple monomer-specific antibody-inducing peptides, each expressed as a multiple antigenic peptide, optionally coupled to carrier protein. Such a construct may be designed employing the MAP system as previously described^{37, 38, 39}. Each MAP contains multiple copies of only one peptide. Therefore, a composition containing MAPs may contain one or multiple different immunogenic peptides. In one embodiment, these MAP constructs are associated with other T cell stimulatory sequences, or as pharmaceutical compositions, administered in conjunction with T cell stimulatory agents, such as known adjuvants.

In another embodiment, a peptide or peptides described above can be part of a self-adjuvanting peptide construct that further comprises in a linear or branched arrangement with the TNF antibody-inducing peptide or peptides, one or more components selected from a promiscuous T helper sequence, a linker sequence, a sequence of charged polar amino acids, and a lipopeptide cap selected from the group consisting of a di- or tri-palmitoyl-S-glyceryl-cysteine. See, e.g., Goldstein, G. and Chicca, J J, Vaccine, 28:1008-1014 (December 2009); International patent publication No. WO2008/021295 and references cited therein, all incorporated herein by reference.

In one embodiment, a single peptide immunogen for inducing antibodies to TNF epitope F comprises the sequence Pam2CSKKKKSQYIKANSKFIGITELGPSLKPWYEPIYLGGVGQLEK SEQ ID NO: 6. The lipopeptide cap is Pam2C; the charged polar amino acids are KKKK; the linkers are S, the T cell helper peptide is QYIKANSKFIGITEL amino acids 8-22 of SEQ ID NO: 6; additional linker amino acids are GPSL, and the immunogenic sequence is KPWYEPIYLGGVFQLEK, amino acids 112-128 of SEQ ID NO: 1. This peptide may be optionally amidated on the carboxy terminus.

In another embodiment, a single peptide immunogen for inducing antibodies to TNF epitope A2 comprises the sequence Pam2CSKKKKSQYIKANSKFIGITELGPSLSRTPSDKPVAHVVANPQAE SEQ ID NO: 7. This immunogen contains the same linkers, lipopeptide cap, T cell helper sequence, polar sequence and linker as the F immunogen, but contains an immunogenic peptide which has the A2 epitope sequence. Again, this peptide immunogen is optionally amidated on the carboxy terminus.

In a similar manner as discussed for immunogens that are not self-adjuvanting, a multi-immunogen that is self-adjuvanting may also be constructed. As one example, a multi-immunogenic construct providing both the A2 and F epitopes has the sequence: Pam2CSKKKKSQYIKANSKFIGITELGPSLSRTPSDKPVAHVVANPQAEGPSLKPWYEPI YLGGVGQLEK SEQ ID NO: 8. As above, the peptide is optionally amidated on the carboxy terminus. In a similar manner, other multi-epitope peptide constructs may be generated by one of skill in the art which meet the criteria described herein.

Still another example of such a self-adjuvanting peptide construct for use herein that contains two TNF antibody-inducing peptides and that can induce antibodies to the three monomer-specific epitopes PSDKPVAH and SSRTPSDKP and EPIYLGGVF has the branched structure:

```
                      RTPSDKPVAHVVA-K₁₅-PWYEPIYLGGVFQLE-amide SEQ ID NO:
                                                      |
Pam2CSKKKKSSQYIKANSKFIGITELS SEQ ID NO: 3
```

The top peptide structure is a fusion of two peptides that can induce antibodies to the two monomer-specific epitopes with a lysine interposed between the peptides at amino acid position 15. The Pam2CSKKKKSSQYIKANSKFIGITELS SEQ ID NO: 3 represents the lipopeptide dipalmitic acid moiety dipalmitoyl-S-glyceryl-Cys (Pam2C), which is attached via a linker, S, to a polar charged sequence, KKKK, to another linker, SS, to a promiscuous T helper sequence naturally found in the tetanus toxin, e.g., QYIKANSKFIGITEL, amino acids 9-23 of SEQ ID NO: 3, to another amino acid linker S. That latter linker amino acid is attached to the epsilon amino group of the lysine in the 15^{th} position (K₁₅) of the fused monomer-specific epitope-containing peptide portion of the construct. As discussed in the cited international patent application above, a variety of options exist for the selection of the components of a self-adjuvanting construct and the branched or linear structure thereof. Thus, one of skill in the art, given this disclosure may readily design a variety of such self-adjuvanting constructs suitable for the presentation of one or more of the three monomer-specific epitopes described herein. Such construction given the present teachings and the prior art are believed to permit a variety of such structures to fall within this invention.

In still another embodiment, one or more of the TNF antibody-inducing peptides, particularly A2 and/or F, described herein can be expressed in a virus-like particle or virosome. See, e.g., Buonaguro L et al, Curr. HIV Res., 2010 June, vol. 8(4): 299-309⁴⁰, incorporated by reference herein. One of more of the immunogenic peptides may be presented in a variety of particulate structures, which allow the insertion or fusion of immunogenic peptide, resulting in a chimeric particle that can deliver the monomer-specific epitope-containing peptide on its surface.

As described above, one of skill in the art, given this disclosure may readily design a variety of such self-adjuvanting constructs suitable for the presentation of one or more of the TNF monomer-specific antibody-inducing peptides described herein. Such construction given the present teachings and the prior art are believed to permit a variety of such structures to fall within this invention.

Preparative techniques for generation of these types of immunogens are well-known in the art and the immunogenic peptides themselves may be generated using the disclosed amino acid sequences of the above-identified epitopes.^{32,36.}

Pharmaceutical Compositions

In another aspect, a pharmaceutical composition comprises one or more of the embodiments described herein of an immunogenic peptide that induces in vivo an antibody that specifically binds to an epitope of a dissociated monomer of human TNF, in a manner in which the binding disrupts or prevents assembly of the monomer into bioactive trimeric human sTNF. The immunogenic compositions containing one or more peptides that induce selective antibodies to a monomer-specific TNF epitope above can be presented in suitable pharmaceutical compositions for presentation to the mammalian, preferably human, subject.

In one embodiment, the pharmaceutical composition contains one of the selective anti-TNF monomer-specific antibody-inducing immunogens described above with a pharmaceutically acceptable carrier, excipient, or diluent. In another embodiment, a pharmaceutical composition comprises two or three selective anti-TNF monomer antibody-inducing immunogens. Thus, the immunogens may induce antibodies to PSDKPVAH and/or PSDKPVAHV and/or EPIYLGGVF. In other embodiment, the pharmaceutical compositions may contain other of the multi-epitope constructs described above. Alternatively, the compositions of this invention may be used in conjunction with, or sequentially with, other therapies or pharmaceutical regimens which are used conventionally to treat the various diseases mediated by sTNF.

These pharmaceutical compositions described herein also contain one or more pharmaceutically acceptable carriers or diluents. As defined herein, the pharmaceutically acceptable carrier suitable for use in an immunogenic proteinaceous composition of the invention are well known to those of skill in the art. Such carriers include, without limitation, and depending upon pH adjustments, buffered water, buffered saline, such as 0.8% saline, phosphate buffer, 0.3% glycine, hyaluronic acid, alcoholic/aqueous solutions, emulsions or suspensions. Other conventionally employed diluents and excipients may be added in accordance with conventional techniques. Optionally, the pharmaceutical compositions can also contain a mild adjuvant, as needed, such as an aluminum salt, e.g., aluminum hydroxide or aluminum phosphate, aqueous suspensions of aluminum and magnesium hydroxides, liposomes, and oil in water emulsions.

Carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, e.g., citrates, ascorbic acid, gluconic acid, carbonic acid, tartaric acid, succinic acid, acetic acid, or phthalic acid, Tris, trimethanmine hydrochloride, or phosphate buffers. Parenteral carriers can include sodium chloride solution, Ringer's dextrose, dextrose and sodium chloride, lactated Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents, inert gases and the like may also be provided in the pharmaceutical carriers. These immunogenic compositions are not limited by the selection of the carrier. The preparation of these pharmaceutically acceptable compositions, from the above-described components, having appropriate pH isotonicity, stability and other conventional characteristics is within the skill of the art. See, e.g., texts such as Remington: The Science and Practice of Pharmacy, 22nd ed, Lippincott Williams & Wilkins, publ., 2012; and The Handbook of Pharmaceutical Excipients, 7th edit., eds. R. C. Rowe et al, Pharmaceutical Press, 2012.

In another embodiment of the pharmaceutical compositions in which the immunogen is a self-adjuvanting construct containing a sequence of charged, polar amino acids that enhances the aqueous solubility of the composition, the composition is formulated with water for injection and optionally a tonicity agent, such as mannitol, without the need for a buffer. In one embodiment, a preferred pharmaceutical carrier contains water for injection with mannitol added for tonicity at a concentration of about 45 mg/mL. The charged polar sequences enable the immunogens to be readily prepared, solubilized and lyophilized.

The amounts of immunogens in the pharmaceutical formulations can vary widely, i.e., from less than about 0.1 mg/mL, usually at or at least about 2 mg/mL to as much as 20 mg/mL, or alternatively up to 50 mg/mL or more by weight, and will be selected primarily by fluid volumes, viscosities, etc., in accordance with the particular mode of administration selected.

A human unit dose form of the immunogenic composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's, cited above.

These compositions may be sterilized by conventional, well known sterilization techniques, such as sterile filtration for biological substances. Resulting aqueous solutions may be packaged for use as is. In certain embodiments in which at least one polar sequence is present in the immunogens of the composition, the aqueous solutions are lyophilized, the lyophilized preparation being combined with a sterile solution prior to administration. Additional pharmaceutically accepted adjuvants may be included, as needed.

As described herein, an advantage of all of the compositions described herein is that none of them induce antibodies that bind transmembrane TNF (tmTNF) or affect the structure or bioactivity of tmTNF.

Methods of Use

Treatment of Certain Inflammatory Conditions

A method for treating a mammalian, preferably human, subject having a disease mediated by soluble human TNF (sTNF) involves reducing the amount, concentration or bioactivity of sTNF in the blood of a subject having the disease without affecting the amount, concentration or bioactivity of tmTNF. This reduction occurs by inducing in vivo in a mammalian subject TNF monomer-specific antibodies that bind an epitope of dissociated monomers of human TNF SEQ ID NO: 1, the epitope being located wholly or partially within a TNF monomer-monomer interface region. The binding disrupts, prevents or reduces the in vivo assembly or reassembly of dissociated monomers of TNF into bioactive trimeric human sTNF.

Thus, in one embodiment, this method comprises administering to a mammalian subject in need thereof an immunogenic composition that comprises a peptide that induces in vivo TNF monomer-specific antibodies that bind an epitope A2 or F of dissociated monomers of human TNF SEQ ID NO: 1, as defined above. In another embodiment, the method involves administering an immunogenic composition that includes an additional or different peptide that induces in vivo TNF monomer-specific antibodies that bind an additional or different epitope of dissociated monomers of human TNF, e.g., a composition that induces both anti-A2 and anti-F antibodies. In the same manner as the first immunogen, the epitope is located wholly or partially within a TNF monomer-monomer interface region, and the binding disrupts in vivo assembly of the monomers into bioactive trimeric human sTNF. In still other embodiments, the immunogenic composition is any one of the immunogenic compositions or pharmaceutical compositions described herein.

The immunogenic compositions contain an effective antibody-inducing amount of each immunogenic anti-A2 or anti-F composition. After a suitable period following administration to a subject, the subject's antiserum contains high affinity, selective polyclonal antibodies that are monomer-specific. The development of such anti-A2 and/or anti-F antibodies in vivo can be monitored, if necessary, by standard techniques, such as with an enzyme-linked immunosorbent assay and surface plasma resonance. The selective, monomer-specific, induced antibodies do not bind intact bioactive sTNF trimer. The selective, monomer-specific, induced antibodies do not bind transmembrane TNF (tmTNF) and do not affect the structure or bioactivity of tmTNF in the immunized subject.

In one embodiment, the method is useful for the treatment of rheumatoid arthritis. In another embodiment, the method is useful for the treatment of ankylosing spondylitis. In another embodiment, the method is useful for the treatment of juvenile rheumatoid arthritis. In still another embodiment, the method is useful for the treatment of psoriatic arthritis. In still another embodiment, the method is useful for the treatment of psoriasisis. In another embodiment, the method is useful for the treatment of a pathogenic effect of bioactive, trimeric sTNF produced during inflammation or during the course of an inflammatory disorder. Still additional embodiments of the methods of the invention involve treatment of other diseases in which sTNF and/or inflammation at low or chronic levels plays a role. In one embodiment, such a disease is HIV-1. In another embodiment, the methods are useful for treating type 2 diabetes. In still other embodiment, the selective, monomer-specific, anti-TNF monomer antibody-inducing immunogenic compositions are useful for treating low level inflammation in the pathology of obesity. In still another embodiment, the selective, monomer-specific, anti-TNF monomer antibody-inducing immunogenic compositions are useful in methods for treating inflammation in the pathology of metabolic syndrome. In another embodiment, the method is useful for the treatment of atherosclerosis and associated cardiovascular disease. In another embodiment, the method is useful for the treatment of inflammation involved in the pathology of Alzheimer's disease. In another embodiment, the method is useful for the treatment of inflammation involved in the pathology of neurodegenerative diseases. Still other inflammatory diseases⁴¹ may be treated with the compositions and methods described herein. Such treatment is not burdened by the immune suppression and morbidity and mortality associated with non-selective therapeutic agents.

Therefore, in one embodiment the immunogenic composition useful in the method induces selective, monomer-specific, anti-TNF monomer antibody that binds the sequence PSDKPVAH or PSDKPVAHV. In another embodiment of the method, the immunogenic composition useful in the method induces selective, monomer-specific, anti-TNF monomer antibody that binds the epitope EPIYLGGVF. The binding of the antibody to the selected epitope disrupts or prevents assembly of the monomer into bioactive trimeric human sTNF. In still further embodiments of this method, the subject is administered both of these selective anti-TNF monomer antibody-inducing immunogenic compositions.

Another aspect of this method involves maintaining a reduced amount or concentration of bioactive trimeric sTNF in the subject's bloodstream over time. Such maintenance can involve repeated or booster administration of one of more of the above-noted immunogenic compositions that induce selective, monomer-specific, anti-TNF antibodies.

These methods have no effect on the amount, concentration or bioactivity of tmTNF in the subject, and therefore the subject's susceptibility to infection by an intracellular pathogen, e.g., tuberculosis, bacterial sepsis, invasive fungal infection, or histoplasmosis, or to a malignancy, e.g., lymphoma or hepatosplenic T-cell lymphoma, is not increased by the administration of these immunogenic compositions.

According to these therapeutic methods, the selective anti-TNF monomer antibody-inducing immunogenic composition is present in a pharmaceutical composition as described above in a pharmaceutically acceptable carrier or diluent. Any of the pharmaceutical compositions described above, e.g., containing one, two or three of the immunogenic peptides that induce selective monomer-specific anti-TNF antibody to a different epitope, and possibly immunogenic peptides that induce antibodies to a selected non-sTNF immunogen, can be employed.

Prevention and Treatment of HIV-1 Infection

In still another aspect, sTNF elevations are also implicated in HIV infection³⁴. In the course of HIV infection, CD4 cells are activated by TNF, and this activation permits the virus to replicate and infect additional cells.

Administration of an immunogenic composition that induces selective anti-TNF antibodies is useful in the prophylaxis of HIV-1 infection. In one embodiment, administration of normal healthy subjects, and especially at-risk populations (e.g., medical and emergency personnel, intravenous drug users and their partners, and persons practicing male on male sex, bisexuals and their partners) with the immunogenic composition is employed to prevent initial TNF increase that accompanies initial HIV-1 infection and acquisition. Such administration can block the induction of activated CD4 cells by TNF, at the outset of infection. Thus these anti-TNF monomer antibody-inducing immunogenic compositions can be used to treat infection in healthy or at-risk patients by preventing, reducing or subduing the initial HIV infection. Alternatively or additionally, the selective anti-TNF monomer antibody-inducing immunogenic compositions are useful in the treatment of HIV-1 infected subjects. Administration of subjects with the immunogenic composition following control of viremia by other means, e.g., anti-retroviral therapy, can prevent or retard reinfection from latently infected cells when anti-retroviral therapy (ART) is withdrawn to permit vaccine control of chronic HIV infection without exposure to adverse reaction related to chronic ART administration. Thus, methods for treating a subject to reduce or prevent re-infection or rebound infection with latent HIV-1 in a subject treated with anti-retroviral drugs use the immunogenic compositions described herein.

Therefore, in still another aspect, a method for treating or preventing HIV-1 infection may also be practiced utilizing the selective anti-TNF monomer antibody-inducing immunogenic compositions and pharmaceutical compositions described herein. In one embodiment, such a prophylactic or therapeutic method involves administering to a subject in need thereof, e.g., a subject in danger of initial HIV-1 infection, a composition that disrupts assembly of the TNF monomers into bioactive trimeric human sTNF, without affecting the amount, concentration or bioactivity of transmembrane TNF.

In one embodiment, the method involves administering an immunogenic composition comprising a peptide that induces in vivo TNF monomer-specific antibodies that bind an epitope (e.g., epitope A2 or epitope F) of dissociated monomers of human TNF SEQ ID NO: 1, the epitope being located wholly or partially within a TNF monomer-monomer interface region, wherein the binding disrupts assembly of the monomers into bioactive trimeric human sTNF.

In another embodiment, the method involves administering a composition containing an additional peptide that induces in vivo TNF monomer-specific antibodies that bind an additional epitope of dissociated monomers of human TNF, the epitope being located wholly or partially within a TNF monomer-monomer interface region, wherein the binding disrupts assembly of the monomers into bioactive trimeric human sTNF., e.g., compositions that induce antibodies against both epitopes A2 and F.

Any of the selective anti-TNF monomer-specific antibody-inducing immunogenic compositions containing peptides that induce antibodies to one or two of the selective monomer-specific epitopes described above, or pharmaceutical composition containing same are useful in this method.

These compositions may be administered optionally in combination with known HIV therapies.

According to any of the above methods, the selective anti-TNF antibody-inducing immunogenic composition is administered in a pharmaceutically acceptable carrier, either separately, in combination, or sequentially in any order. The routes of administration and dosages are anticipated to be as described. However, one of skill in the art, given the teachings of this application may employ other suitable dosages and routes of administration.

Treatment of Diabetes

In still another aspect, sTNF elevations are also implicated in type II diabetes. Yet a further embodiment involves a method for treating diabetes, which may also be practiced utilizing the anti-TNF monomer antibody-inducing immunogenic compositions and pharmaceutical compositions described herein. In one embodiment, this method for treating a human subject with type II diabetes comprises administering periodically to a subject in need thereof one of the anti-TNF monomer antibody-inducing immunogenic compositions or pharmaceutical composition. These compositions may be any of those described specifically above. In one embodiment, the subject is concurrently treated with other diabetes medication. In still another embodiment, the administration of the selective anti-TNF monomer antibody-inducing immunogenic composition is repeated periodically after the subject ceases treatment with other diabetes medications, such as insulin or oral drugs such as metformin.

Modes of Administration and Dosages

The methods described above involve administering to a subject an effective antibody-inducing amount of the immunogenic and pharmaceutical compositions described herein.

In one embodiment of this method, the route of administration of these immunogenic and pharmaceutical compositions is subcutaneous injection. In another embodiment of this method, the route of administration of these immunogenic and pharmaceutical compositions is intravenous injection. In another embodiment of this method, the route of administration of these immunogenic and pharmaceutical compositions is intramuscular injection. In still other embodiments of each of the above-described methods, these compositions of the present invention are administered by an appropriate route, e.g., mucosal, intra-arterial, intraperitoneal, parenteral, intradermal, transdermal, nasal, vaginal, or rectal or inhalation routes.

The appropriate route is selected depending on a variety of considerations, including the nature of the composition, i.e., as a prophylactic immunogenic composition or as a therapeutic, and an evaluation of the age, weight, sex and general health of the patient and the components present in the immunogenic composition, and similar factors by an attending physician.

Suitable doses of the immunogenic compositions and self-adjuvanting immunogenic compositions used in the methods above are readily determined by one of skill in the art, whether the patient is already infected and requires therapeutic treatment or prophylactic immunogenic composition treatment, the health, age and weight of the patient. The amount of the immunogenic peptides that induce a selective anti-TNF monomer antibody described above, with or without other immunogens, present in each dose, is selected with regard to consideration of the patient's age, weight, sex, general physical condition and the specific disease being treated. The amount of immunogenic peptide required to produce an exogenous effect in the patient without significant adverse side effects varies depending upon the pharmaceutical composition employed.

When used together, dosages of each immunogenic peptide that induces a monomer-specific anti-TNF monomer antibody to a different one of the TNF epitopes may be the same. In another embodiment, due to the synergy between the two combined selected immunogenic peptides, a combination dosage is lower than additive single dosages of each antibody alone. Additional combination with immunogenic peptides that induce antibodies directed to other than TNF monomer-specific epitopes may alter the dosage of the anti-TNF monomer-specific antibody-inducing peptides.

In one embodiment, the administration of the selective anti-TNF monomer antibody-inducing immunogenic composition is repeated periodically during the course of the disease or during the course of the subject's exposure to infection, or during the course of a chronic disease. In various embodiments in which one or both of the selective anti-TNF monomer antibody-inducing immunogenic peptides or compositions are administered in the course of treatment, each immunogenic peptide in a pharmaceutically acceptable carrier is administered, either separately, in combination, or sequentially in any order.

The method and routes of administration and the presence of additional components in the compositions may also affect the dosages and amounts of the compositions. Such selection and upward or downward adjustment of the effective dose is within the skill of the art. The amount of composition required to produce a suitable response in the patient without significant adverse side effects varies depending upon these factors. Suitable doses are readily determined by persons skilled in the art. A suitable dose of an immunogenic composition described herein is formulated in a pharmaceutical composition, as described above (e.g., dissolved in about 0.1 mL to about 2 mL of a physiologically compatible carrier) and delivered by any suitable means. Dosages are typically expressed in a “unit dosage”, which is defined as dose per subject, e.g., a unit dosage of 1 mg immunogen. Alternatively dosages can be expressed as amount per body weight of the subject or patient, using the norm for therapeutic conversions as 80 kg body weight. For example, a 1 mg unit dose per subject is equivalent to about 12.5 μg/kg body weight.

In one embodiment, the intended therapeutic or prophylactic effect of any of the methods is conferred by a priming/boosting dosing regimen. For example, any of the methods described above comprises administering to the subject an initial or priming effective amount of the immunogenic compositions. In one embodiment, the dosage for an initial therapeutic administration or for a first priming therapeutic or prophylactic immunogenic composition administration in one embodiment is a “unit dosage” of less than about 0.01 mg to 100 mg of immunogen. In one embodiment, the unit dosage is 0.01 mg. In another embodiment, the unit dosage is 0.1 mg. In another embodiment, the unit dosage is 1 mg. In still another embodiment, the unit dosage is 10 mg. Thus, the initial priming dosage for a human, in certain embodiments, can range from very low unit dosages of at least about 0.01, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, to higher dosages of at least 1 mg, at least 3 mg, at least 5 mg, at least 7 mg, at least 10 mg, at least 12 mg, at least 15 mg, at least 20 mg. Still other human dosages range from between 21-30 mg, 31-40 mg, 41-50 mg, 51-60 mg, 61-70 mg, 71-80 mg, 81-90 mg and 91-100 mg/70-80 kg subject. Even higher dosages may be contemplated.

Other embodiments of the methods described herein include administering at least one booster effective amount of the immunogenic composition. In one embodiment, the boosting dosages for either therapeutic prophylactic immunogenic composition or prophylactic immunogenic composition use are the same as the above described priming dosage. The same specific unit dosage or unit dosage ranges as for the priming dosage above may be employed for the boosting dosage. Thus, the boosting dosage for a human, in certain embodiments, can occur in a unit dosage range a “unit dosage” of less than about 0.01 mg to 100 mg of immunogen. In one embodiment, the unit dosage is 0.1 mg. In another embodiment, the unit dosage is 1 mg. In still another embodiment, the unit dosage is 10 mg. Thus, the booster unit dosage for a human, in certain embodiments, can range from very low unit dosages of at least about 0.01, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, to higher dosages of at least 1 mg, at least 3 mg, at least 5 mg, at least 7 mg, at least 10 mg, at least 12 mg, at least 15 mg, at least 20 mg. Still other human dosages range from between 21-30 mg, 31-40 mg, 41-50 mg, 51-60 mg, 61-70 mg, 71-80 mgs, 81-90 mg and 91-100 mg/70-80 kg subject. Even higher dosages may be contemplated. In alternative embodiments, the boosting dosages are lower than the priming dosage identified above.

In one embodiment, the first “boosting” is administered within weeks of the initial priming dose. In one embodiment, the boosting dose is administered at least 3 weeks after the priming dose, followed by a re-boost administered not earlier than 3 weeks from the preceding boosting dose. In another embodiment, the first boosting dose is administered about 3 to 4 weeks following the priming dose. Additional boosting dosages are administered thereafter at least 3 weeks thereafter, more suitably about 6 months to one or more years, following the first booster dose. In another embodiment of an administration protocol, a priming dosage of a self-adjuvanting immunogenic composition as described herein is administered which is about 10 mg. The subsequent first boosting dosage (e.g., 0.01, 0.1, 1 or 10 mg) is then administered at least three weeks after the priming dosage. Thereafter, additional boosting dosages are administered every 6 months to one year from the preceding boosting dosage.

The timing and dosage of any priming/boosting regimen may be selected by the attending physician depending upon the patient's response and condition as determined by measuring the specific monomer-specific anti-TNF monomer antibody titer obtained from the patient's blood, as well as normal considerations related to the physical condition of the patient, e.g., height, weight, age, general physical health, other medications, etc. Similarly, in the method combining administration of the anti-TNF monomer antibody-inducing immunogenic composition with the anti-HIV-1 Tat antibody-inducing immunogenic composition, the timing and dosage depends upon similar determination of antibody titer in the immunized subject.

In one embodiment of the prophylactic/therapeutic method involves administering a priming effective amount of the immunogenic composition in a unit dosage of less than or about 10 mg, 1 mg or 0.1 mg, and following up the administration by two boosters administered at weeks 3 and weeks 6 at the same effective unit dosage.

Administration desirably continues until at least clinical symptoms or laboratory tests indicate that the disease or infection has been eliminated or reduced in severity or activity, and for a period thereafter. The dosages, routes of administration, and dose schedules are adjusted in accordance with methodologies known in the art.

For prophylactic immunogenic composition use, the priming and boosting dosages are similar to the boosting dosages of the therapeutic mmunogenic composition, but are administered at certain defined intervals from about two weeks to six months after the initial administration of prophylactic immunogenic composition. Possibly additional prophylactic immunogenic composition administrations may be desirable thereafter.

The following examples illustrate certain embodiments of the above-discussed compositions and methods. These examples do not limit the disclosure of the claims and specification.

### EXAMPLES

The following examples illustrate certain embodiments of the above-discussed compositions and methods. These examples do not limit the disclosure of the claims and specification.

### Example 1

### TNF Epitope Mapping

The 157 amino acid TNF monomers SEQ ID NO: 1 have an elongated, anti-parallel β pleated sheet structure. When three monomers are associated in a non-covalent trimer, bioactive sTNF is formed. Five stretches of amino acid sequences form the interface β sheet contact surfaces:

| | |
|---|---|
| A | KPVAHVVA, aa11-18 of SEQ ID NO: 1; |
| A′ | ALLAN, aa35-39 of SEQ ID NO: 1; |
| C | GLYLIYSQVLFKGQ, aa54-67 of SEQ ID NO: 1; |
| F | WYEPIYLGGVFQL, aa114-126 of SEQ ID NO: 1; |
| | and |
| H | QVYFGIIAL, aa149-157 of SEQ ID NO: 1, |

where A, A′, C, F and H refer to a β sheet naming convention¹¹.

To attain trimer disruption immunologically, the inventor theorized that antibody binding to epitope sequences that are wholly or partially within the contact area between adjacent monomers (the so-called internal or interface regions) would not bind to intact trimers of sTNF or tmTNF but would only bind to free monomers of TNF. In binding only to the free monomers, these antibodies would disrupt or prevent the ability of the monomers to re-associate and form active trimers.

Therefore, the inventor explored all five regions for potential B cell epitopes and detected and mapped antibodies to two epitopes partially (the A interface β sheet contact surface) and one epitope fully (the F interface β sheet contact surface) within an interface region. Rats or mice were immunized with synthetic peptides derived from the linear sequence from the five known interface regions of TNF identified above⁵. These synthetic peptides sequences were conjugated with KLH, and adjuvants as needed such as Freunds Complete or Incomplete Adjuvant were used. Alternatively the synthetic peptide sequences are incorporated in self adjuvanting constructs, such as those described for HIV Tat constructs.³³ Polyclonal antibodies isolated from rats immunized with each of the synthetic peptide sequences were evaluated on rTNF and also on the synthetic TNF peptides using conventional ELISAs. See, e.g., the protocols described for anti-human TNF/TNFSF1A antibody by R&D Systems, catalog number MAB610, clones 28401, pages 1 and 2 (Jun. 17, 2005).

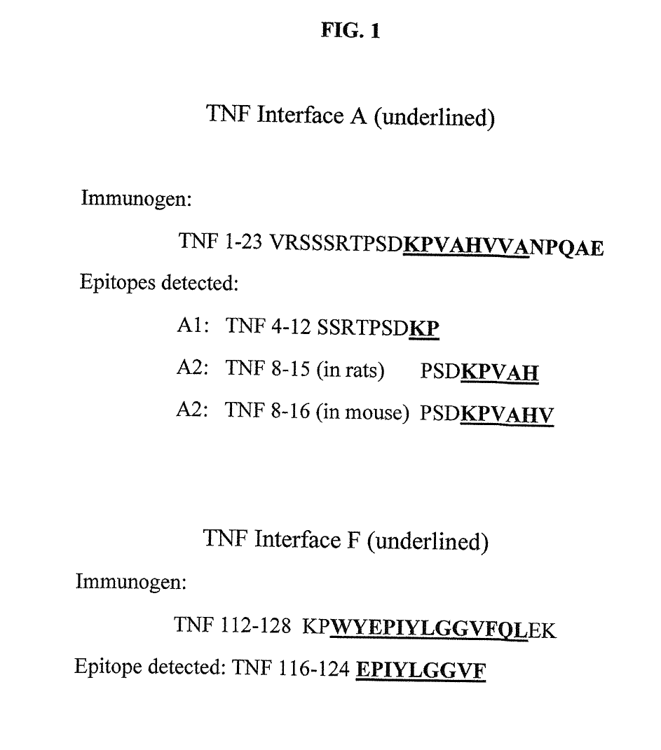

Binding to truncated sequences from larger peptide immunogens was used to delineate epitope margins. When antibodies were detected to a region of TNF, truncated peptide sequences were used to determine the margins of the epitopes defined above. An antibody that bound exclusively to one of each the specific epitope sequences was referred to as a selective anti-TNF monomer antibody of this invention. See, e.g., FIG. 1.

Table 1 sets out the interface regions, immunogens tested and epitopes detected from the epitope searches:

TABLE 1

| | Immunogens | | |
|---|---|---|---|
| Interface | of SEQ ID NO: | | |
| Region | 1 Tested | Epitopes Detected* | Titers |
| A | aa1-15 and | aa4-12: SSRTPSDKP | 400,000 to |
| | aa1-24 | (epitope A1) | 1 million |
| | | aa8-15: PSDKPVAH | |
| | | (epitope A2) | |
| A′ | aa35-39 | No significant | — |
| | | antibody response | |
| C | aa52-67 | No significant | — |
| | | antibody response | |
| F | aa112-128 | aa116-124: EPIYLGGVF | 50,000 to |
| | | (epitope F) | 100,000 |
| H | aa145-157 | No significant | — |
| | | antibody response | |

*Bold amino acids within interface sequences

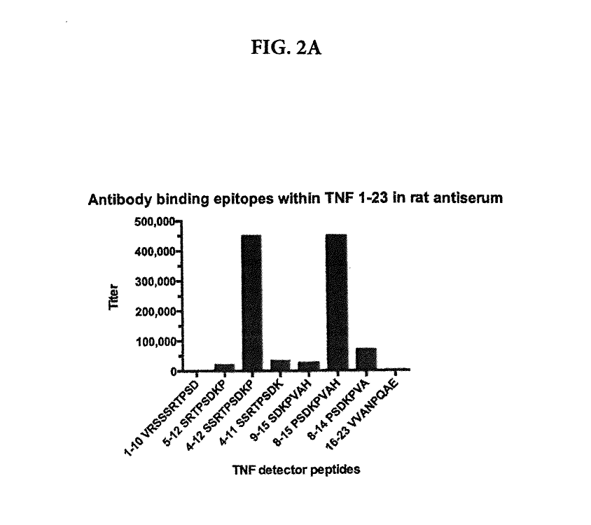

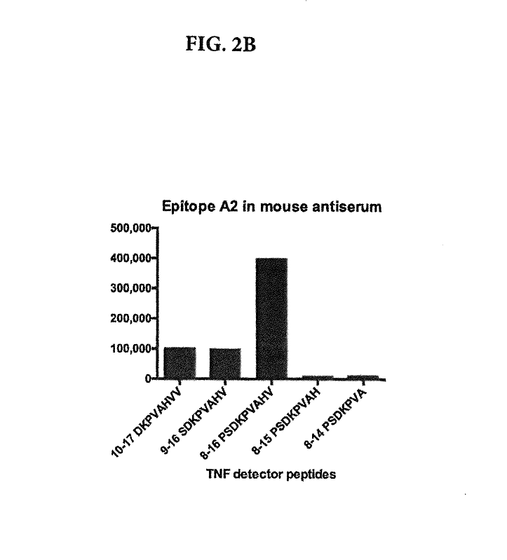

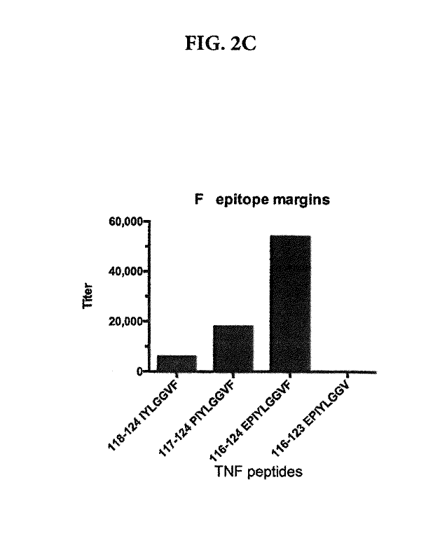

FIGS. 2A, 2B and 2C illustrate the margin identification of the epitopes.

All three epitope sequences are outside of the known regions of the receptor binding sites of TNF^{12,13,15}. The two overlapping B cell epitopes detected in synthetic peptide sequences overlapping interface contact region A are:
PSDKPVAH, amino acids 8-15 of SEQ ID NO: 1 and PSDKPVAHV, amino acids 8-16 of SEQ ID NO: 1; and
SSRTPSDKP, amino acids 4-12 of SEQ ID NO: 1.

Thus, the TNF aa1-23 monomer sequence of SEQ ID NO: 1 contains only two B cell epitopes, each epitope containing interface amino acid(s) essential for antibody binding, but only epitope A2 contains amino acids necessary for trimer formation and biological function.

The entire sequence of the third epitope EPIYLGGVF (F sheet), amino acids 116-124 of SEQ ID NO: 1 was within interface contact region. F. Two of the epitope amino acids, Tyr₁₁₉ and Gly₁₂₂, are critical for trimer formation and biological activity^{13,24}.

In the three epitopes shown above, the bolded amino acids are within the internal interface regions. The italicized amino acids are those at which point mutations induce failure to form trimers and loss of biological activity.

Antibodies to each of three epitopes A1: SSRTPSDKP, A2: PSDKPVAH/PSDKVPAHV and F: EPIYLGGVF bind to synthetic rTNF (which comprises trimer, dimer and monomer forms) in the conventional ELISA assay. Antibodies to A1 SSRTPSDKP (both polyclonal antibodies and monoclonal antibodies) bound only two interface amino acids (Lys₁₁ and Pro₁₂), essential for antibody binding to the epitope. Neither of these amino acids have been shown to be critical for trimer formation and bioactivity of sTNF. Antibodies to A2 PSDKPVAH or PSDKPVAHV (both polyclonal antibodies and monoclonal antibodies) also masked His₁₅ that is critical to trimer formation and bioactivity of sTNF molecules^{13,23}.

Only one other monomer specific antibody was detected, within the F monomer interface region. The F epitope spanned TNF amino acids 116-124 and induced polyclonal antibodies that were monomer-specific. These antibodies masked Tyr₁₁₉ and Gly₁₂₂, both critical to trimer formation and bioactivity of sTNF molecules.

### Example 2

### Selective Binding Activity

Natural or synthetic sTNF consists of a mixture of inactive TNF monomers, inactive TNF dimers and bioactive TNF trimers. Each antibody used in the assays and commercially available anti-TNF antibodies (e.g., REMICADE) or commercially available TNF receptor chimera (e.g., ENBREL) binds to synthetic TNF coated on a plate, as in the conventional ELISAs performed in Example 1. However, detection of specific binding of an antibody/ligand to the monomeric, dimeric or trimeric form of TNF requires appropriate selective assays. To demonstrate the selective binding activity of antibodies or ligands that bind only the three epitopes on a monomer as identified in Example 1, the following assays are performed:

Sandwich Assay

In one embodiment, a sandwich assay employs a biotinylated anti-TNF monomer-specific antibody which binds the sTNF in a sample, followed by detection with the same antibody, non-biotinylated, coated on a plate²⁵. The unlabeled antibody is plated (e.g., one of the inventor's epitope-binding antibodies or a commercial antibody, e.g., REMICADE antibody). A sample containing synthetic sTNF mixed with the same anti-TNF monomer-specific antibody, which has been biotinylated, is prepared and then introduced to the plate. The plate is then washed and any bound sTNF sandwiched between the unlabeled bound antibody and the labeled detector antibody is measured using a suitable detector system, e.g., streptavidin/horseradish peroxidase, to generate a detectable signal.

If the antibody used as the capture/detector antibody is an antibody that binds TNF trimeric form, a sandwich effect will be detected. Only TNF trimers with the ability to simultaneously bind both the plate-bound antibody and the biotinylated antibody will demonstrate binding in this assay. This is because in the mixture not all binding sites on the trimers will be bound, thereby leaving extra binding sites to be captured on the plate. For example, the commercial REMICADE antibody demonstrates trimer binding in this assay.

In contrast, the inventor's antibodies that specifically bind a single epitope on a sTNF monomer do not demonstrate binding in this assay. Once each monomer is bound by the labeled antibody in the mixture, that epitope is no longer available for binding to the same unlabeled antibody on the plate when the mixture is added to the plate. A sandwich cannot form.

This assay can therefore be used to distinguish between the anti-TNF antibodies/ligands that bind to A2 or F selectively and that bind only TNF monomers and those non-selective commercial and known anti-TNF antibodies that bind trimers, both tmTNF and trimeric sTNF.

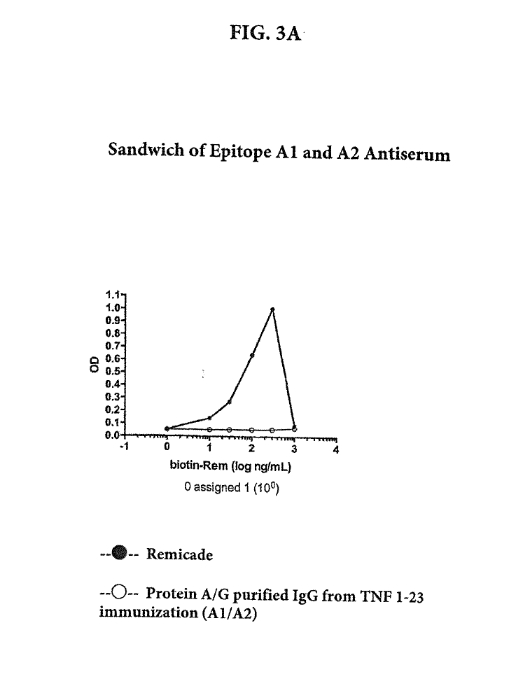

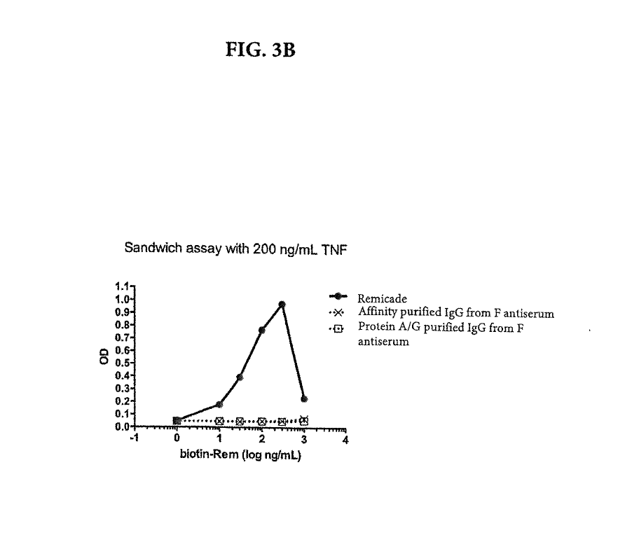

When employed in these assays, the inventor determined that antibodies (both polyclonal and monoclonal) to epitopes A1, A2 or F all were able to bind only TNF monomers. Results from performance of this assay are shown in FIGS. 3A and 3B.

Functional Assay

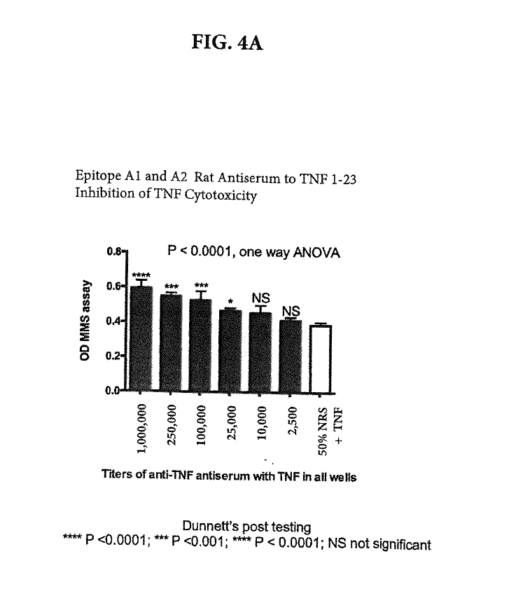

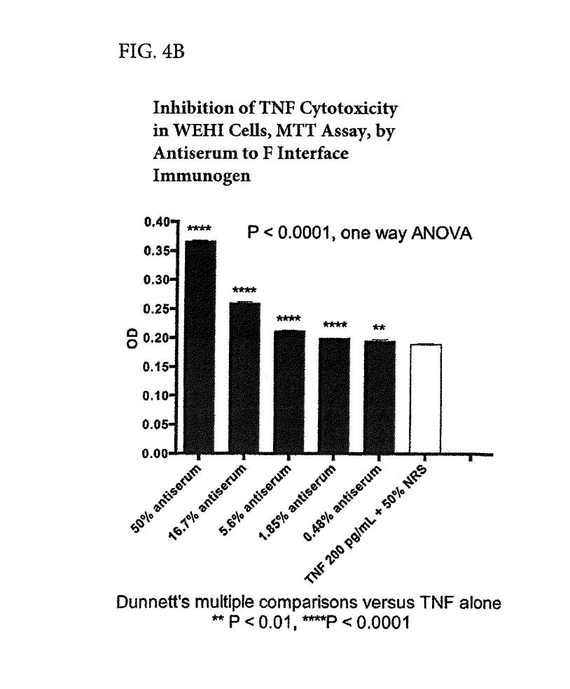

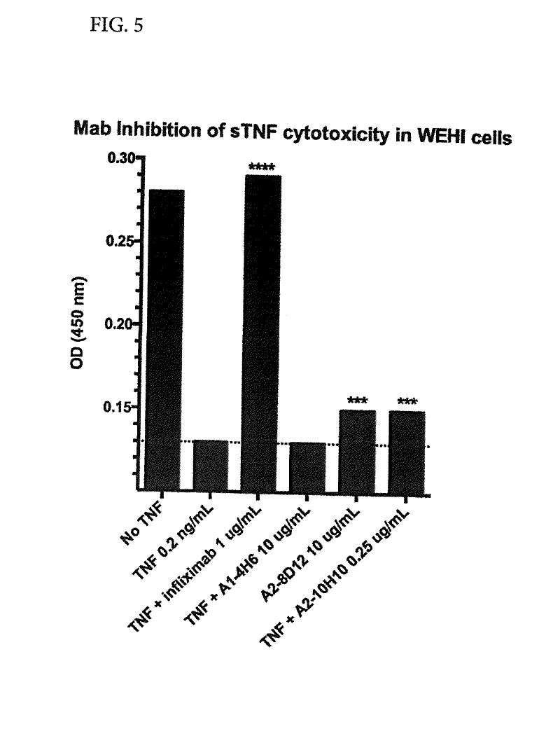

A functional assay is one that demonstrates the effects of the antibodies on TNF binding cells, such as actinomycin treated WEHI cells. See, e.g., the protocols for “Neutralization of Human TNF Bioactivity” described for anti-human TNF/TNFSF1A antibody by R&D Systems, catalog number MAB610, clones 28401, pages 1 and 2 (Jun. 17, 2005), incorporated by reference herein. For the generation of the data in FIGS. 4B and 5, in place of the cells L929, WEHI cells are used. This assay showed inhibition of TNF activity by the selected tested antibodies. In this assay the antibodies that bind one of the three epitopes on the monomers as described herein are evaluated to determine if they have the ability to disrupt the formation of bioactive trimers of TNF and thus inhibit TNF activity in a dose response curve. Performance of this functional assay and its results are demonstrated in FIGS. 4B and 5.

Results

In the sandwich assay described above, the selective antibodies that bound one of the three epitopes on the TNF monomer: A1: SSRTPSDKP, A2:PSDKPVAH or PSDKPVAHV, and F: EPIYLGGVF, bound only the monomeric form of sTNF. As described above, the biotinylated selective anti-TNF antibodies that bind one of the epitopes A1, A2 or F would not sandwich with sTNF to cause binding by the same selective non-biotinylated anti-TNF monomer antibody coated plate. See, e.g., FIGS. 3A and 3B. In contrast to the results for the anti-A2 and anti-F antibodies/ligands, the commercial anti-TNF REMICADE antibody demonstrated binding in the sandwich assay (see e.g., FIG. 3), showing binding to trimeric TNF. Additionally all polyclonal antibodies to TNF peptides encroaching an interface region were negative in the sandwich assay. These data show a lack of binding to trimers, demonstrating that binding was restricted to monomers.

However, only two of the antibodies that exhibited selective binding to one of the three epitopes, A1: SSRTPSDKP, A2: PSDKPVAH and F: EPIYLGGVF produced dose-responsive inhibition of TNF, suppression of TNF inhibition of cell replication in actinomycin treated WEHI cells in the functional assay. See, e.g., FIGS. 4A, 4B and 5. Antibodies to A2 and F were capable of disrupting trimer formation and were associated with inhibition of sTNF binding to TNF receptors and inhibition of cytotoxicity of sTNF on actinomycin treated WEHI cells. These assay findings provided evidence that the monomer specific antibodies to epitopes A2 and F disrupt trimer assembly by blocking specific amino acid side chains essential for inter-monomer binding.

Surprisingly, antibodies to the A1 epitope, while monomer specific, were inactive in blocking TNF function.

### Example 3

### Generation of an Immunogenic Composition

Experimental Immunogens

Various immunogenic compositions as described above were prepared containing an immunogenic peptide that induces in vivo TNF monomer-specific antibodies that binds an epitope of dissociated monomers of human TNF of this invention.

One example of an immunogen that induces anti-F epitope antibodies is C-KPWYEPIYLGGVFQLEK SEQ ID NO: 9, which optionally is amidated on the carboxy terminus. An example of an immunogen inducing anti A2 and anti F epitopes has the sequence: C-SRTPSDKPVAHVVANPQAEGPSLKPWYEPIYLGGVGQLEK SEQ ID NO: 5, which may have a free amide on the carboxy terminus. These immunogens are formulated with an adjuvant.

Other examples of useful immunogens present the TNF epitopes in a self-adjuvanting construct. One such exemplary construct contains two different TNF monomer specific peptides. The other components of the construct are a T cell helper sequence, linker amino acids, a polar sequence and the Pam2C-lipoprotein cap according to the formula SEQ ID NO: 6: Pam2CSKKKKSQYIKANSKFIGITELGPSLKPWYEPIYLGGVGQLEK, wherein the peptide is optionally amidated on the carboxy terminus.

Still another example of an immunogenic comprises the sequence SEQ ID NO: 7: Pam2CSKKKKSQYIKANSKFIGITELGPSLSRTPSDKPVAHVVANPQAE, wherein the peptide is optionally amidated on the carboxy terminus.

Another exemplary construct has the sequence SEQ ID NO: 8: Pam2CSKKKKSQYIKANSKFIGITELGPSLSRTPSDKPVAHVVANPQAEGPSLKPWYEP IYLGGVGQLEK, wherein the peptide is optionally amidated on the carboxy terminus.

Still another exemplary construct contains two different TNF monomer-specific peptides. The other components of the construct are a T cell helper sequence (italicized and bolded), linker amino acids (italicized only), a polar sequence (bolded) and the Pam2C-lipoprotein cap according to the formula below

```
                            SRTPSDKPVAH-K₁₅-PWYEPIYLGGVFQLE-amide SEQ ID NO: 2
                                                      |
Pam2CSKKKKSS [symbol] S SEQ ID NO: 3
```

The lipopeptide cap/T helper sequence SEQ ID NO: 3 is attached to the epsilon amino group of the lysine interposed at amino acid position 15 (K₁₅) between the two fused sTNF immunogenic peptides.

The Pam2C- and Pam3C immunogens prepared according to the preceding formulae are synthesized by Bachem Biosciences, Inc. or Anaspec, Inc. using conventional solid phase synthesis techniques and automated synthesizers. Alternative immunogens are prepared in a similar manner which contain totally linear sequences, different lipopeptide or T helper components, linkers or polar sequences, or in which the components arranged in a different linear or branched structure. Still other alternative immunogens are prepared containing a single TNF monomer-specific peptide immunogen.

In either synthesis, Fmoc protected di-palmitoyl-S-glyceryl-cysteine is synthesized by Bachem and is coupled to the N-terminal serine of the peptide chain containing the T helper sequence as indicated. Trifluoroacetic acid is used to cleave the lipopeptide from the resin and deprotect the peptide. The resulting immunogenic product is dried and then taken into aqueous solution, converted to an acetate salt form and dried.

The final product is checked by amino acid analysis for the appropriate content of amino acids, and by mass spectroscopy. Purity is estimated in general to be around 70% for each construct, but the resulting lipopeptides are not purified further.

### Example 4

### Immunization Protocols

Immunizations

Animals are purchased from Harlan Laboratories and acclimated at Molecular Diagnostic Services, Inc. for at least 1 week before immunization. Both BALBc and C57BL6/BALBc F1 mice are used. Lewis rats are used as indicated.

Immunogens prepared as described in Example 3 are taken up in buffered saline and injected IP in mice and rats, and SC in rabbits. Unless otherwise stated, mice are immunized IP with 1 mg of Immunogen at Day 0 and Week 2 with complete Freunds adjuvant and then at 2 weekly intervals in Incomplete Freunds adjuvant. Two weeks after the last boost they are bled for serum analysis.

Note that the tet toxoid T cell helper sequence used in the immunogens discussed herein was originally discovered as having promiscuous helper activity in human cells and appears to have wide activity in multiple animal species.

Serum Titration

Serums are assayed to determine monomer-specific anti-TNF titers (and possibly anti-Tat titers) by conventional ELISA methods. See, e.g., the protocols described in Examples 1 and 2 and in the examples of International patent publication No. WO2008/021295, incorporated by reference herein. See, also, FIGS. 4A and 4B.

Bioassays demonstrating inhibition of TNF activity are conducted as described in Example 2. Bioassay of Tat activity and inhibition by antisera from immunized animals are conducted as described in conventional assays as described previously, e.g., in International patent publication No. WO2008/021295.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunogenic peptide” is understood to represent one or more immunogenic peptides. As such, the terms “a” (or “an”), “one or more,” and “at least one” is used interchangeably herein.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application. All documents listed or referred to herein, including U.S. Provisional application Nos. 61/756,583 and 61/768,055 as well as the attached or electronic Sequence Listing, are incorporated herein by reference.

Without further description, it is believed that one of ordinary skill in the art can, using the preceding description and the following illustrative examples, make and utilize the compositions of the present invention and practice the claimed methods. While the invention has been described and illustrated herein by references to various specific materials, procedures and examples, it is understood that the invention is not restricted to the particular combinations of material and procedures selected for that purpose.

Numerous modifications and variations of the embodiments illustrated above are included in this specification and are expected to be obvious to one of skill in the art. Such modifications and alterations to the compositions and processes described herein are believed to be encompassed in the scope of the claims appended hereto.

The following information is provided for sequences containing free text under numeric identifier <223>.

TABLE 2
(Sequence Listing Free Text)

| SEQ ID NO: | |
|---|---|
| (containing free text) | Free text under <223> |
| 2 | Misc_Feature: residue 15 is |
| | modified at epsilon amino group |
| | by linking to T helper peptide |
| | sequence 37 modified with a lipid |
| 2 | Misc_Feature: residue 30 can be |
| | modified with an amide |
| 3 | Misc_Feature: residue 1 is |
| | modified with the lipid, |
| | dipalmitoyl-S-glyceryl |
| 6 | Misc_Feature: residue 1 is |
| | modified with the lipid, |
| | dipalmitoyl-S-glyceryl |
| 7 | Misc_Feature: residue 1 is |
| | modified with the lipid, |
| | dipalmitoyl-S-glyceryl |
| 8 | Misc_Feature: residue 1 is |
| | modified with the lipid, |
| | dipalmitoyl-S-glyceryl |

### REFERENCES

1. Taylor, P. C. & Feldmann, M. Anti-TNF biologic agents: still the therapy of choice for rheumatoid arthritis. Nature reviews. Rheumatology 5, 578-582 (2009).
2. Feldmann, M. & Maini, R. N. Anti-TNF therapy, from rationale to standard of care: what lessons has it taught us? J Immunol 185, 791-794 (2010).
3. Wallis, R. S., Broder, M. S., Wong, J. Y., Hanson, M. E. & Beenhouwer, D. O. Granulomatous infectious diseases associated with tumor necrosis factor antagonists. Clinical infectious diseases: an official publication of the Infectious Diseases Society of America 38, 1261-1265 (2004).
4. Bongartz, T., et al. Anti-TNF antibody therapy in rheumatoid arthritis and the risk of serious infections and malignancies: systematic review and meta-analysis of rare harmful effects in randomized controlled trials. JAMA: the journal of the American Medical Association 295, 2275-2285 (2006).
5. Scallon, B. J., Moore, M. A., Trinh, H., Knight, D. M. & Ghrayeb, J. Chimeric anti-TNF-alpha monoclonal antibody cA2 binds recombinant transmembrane TNF-alpha and activates immune effector functions. Cytokine 7, 251-259 (1995).
6. Mitoma, H., et al. Infliximab induces potent anti-inflammatory responses by outside-to-inside signals through transmembrane TNF-alpha. Gastroenterology 128, 376-392 (2005).
7. Olleros, M. L., et al. Transmembrane TNF induces an efficient cell-mediated immunity and resistance to Mycobacterium bovis bacillus Calmette-Guerin infection in the absence of secreted TNF and lymphotoxin-alpha. J Immunol 168, 3394-3401 (2002).
8. Li, Q., et al. Mechanism of action differences in the antitumor effects of transmembrane and secretory tumor necrosis factor-alpha in vitro and in vivo. Cancer immunology, immunotherapy: CII 55, 1470-1479 (2006).
9. Alexopoulou, L., et al. Transmembrane TNF protects mutant mice against intracellular bacterial infections, chronic inflammation and autoimmunity. European journal of immunology 36, 2768-2780 (2006).
10. Smith, R. A. & Baglioni, C. The active form of tumor necrosis factor is a trimer. The Journal of biological chemistry 262, 6951-6954 (1987).
11. Eck, M. J. & Sprang, S. R. The structure of tumor necrosis factor-alpha at 2.6 A resolution. Implications for receptor binding. The Journal of biological chemistry 264, 17595-17605 (1989).
12. Van Ostade, X., Tavernier, J., Prange, T. & Fiers, W. Localization of the active site of human tumour necrosis factor (hTNF) by mutational analysis. The EMBO journal 10, 827-836 (1991).
13. Zhang, X. M., Weber, I. & Chen, M. J. Site-directed mutational analysis of human tumor necrosis factor-alpha receptor binding site and structure-functional relationship. The Journal of biological chemistry 267, 24069-24075 (1992).
14. Yang, Z., West, A. P., Jr. & Bjorkman, P. J. Crystal structure of TNFalpha complexed with a poxvirus MHC-related TNF binding protein. Nature structural & molecular biology 16, 1189-1191 (2009).
15. Mukai, Y., et al. Solution of the structure of the TNF-TNFR2 complex. Science signaling 3, ra83 (2010).
16. Tang, P., Hung, M. C. & Klostergaard, J. Human pro-tumor necrosis factor is a homotrimer. Biochemistry 35, 8216-8225 (1996).
17. Utsumi, T., et al. Transmembrane TNF (pro-TNF) is palmitoylated. FEBS letters 500, 1-6 (2001).
18. Corti, A., Fassina, G., Marcucci, F., Barbanti, E. & Cassani, G. Oligomeric tumour necrosis factor alpha slowly converts into inactive forms at bioactive levels. The Biochemical journal 284 (Pt 3), 905-910 (1992).
19. Steed, P. M., et al. Inactivation of TNF signaling by rationally designed dominant-negative TNF variants. Science 301, 1895-1898 (2003).
20. Zalevsky, J., et al. Dominant-negative inhibitors of soluble TNF attenuate experimental arthritis without suppressing innate immunity to infection. J Immunol 179, 1872-1883 (2007).
21. He, M. M., et al. Small-molecule inhibition of TNF-alpha. Science 310, 1022-1025 (2005).
22. Wells, J. A. & McClendon, C. L. Reaching for high-hanging fruit in drug discovery at protein-protein interfaces. Nature 450, 1001-1009 (2007).
23. Yamamoto, R., Wang, A., Vitt, C. R. & Lin, L. S. Histidine-15: an important role in the cytotoxic activity of human tumor necrosis factor. Protein engineering 2, 553-558 (1989).
24. Douni, E., et al. A RANKL G278R mutation causing osteopetrosis identifies a functional amino acid essential for trimer assembly in RANKL and TNF. Human molecular genetics 21, 784-798 (2012).
25. Nagahira, K., et al. Epitope mapping of monoclonal antibodies to tumor necrosis factor-alpha by synthetic peptide approach. Immunology letters 46, 135-141 (1995).
26. Socher, S. H., et al. Antibodies against amino acids 1-15 of tumor necrosis factor block its binding to cell-surface receptor. Proceedings of the National Academy of Sciences of the United States of America 84, 8829-8833 (1987).
27. Chackerian, B., Lowy, D. R. & Schiller, J. T. Conjugation of a self-antigen to papillomavirus-like particles allows for efficient induction of protective autoantibodies. The Journal of clinical investigation 108, 415-423 (2001).
28. Spohn, G., et al. A virus-like particle-based vaccine selectively targeting soluble TNF-alpha protects from arthritis without inducing reactivation of latent tuberculosis. J Immunol 178, 7450-7457 (2007).
30. Fee, C. J., Protein conjugates purification and characterization, PEGylated Protein Drugs: Basic Science and Clinical Applications, Veronese, F. M., Ed. Birkhauser Publishing: Basel, 113-125 (2009).
31. Fee, C. J.; Damodaran, V. B., Production of PEGylated Proteins, in Biopharmaceutical Production Technology, G. Subramanian, Ed. Wiley-VCH Verlag GmbH & Co. KGaA, Weinheim, Germany (2012).
32. Harlow, E. and Lane, D. (1988). Antibodies: A Laboratory Manual. Cold Spring Harbor, N.Y.: Cold Spring Harbor Laboratory.
33. Goldstein, G. and Chicca, J J, A universal anti-HIV-1 Tat epitope vaccine that is fully synthetic and self-adjuvanting vaccine., Vaccine, 28:1008-1014 (December 2009).
34. Stacy, A R et al, Induction of a Striking Systemic Cytokine Cascade prior to Peak Viremia in Acute Human Immunodeficiency Virus Type 1 Infection, in Contrast to More Modest and Delayed Responses in Acute Hepatitis B and C Virus Infections, J. Virol., 83(8):3719-3733 (April 2009)
35. International patent publication No. WO2008/021295, published February 2008, entitled “Compositions And Methods For The Treatment And Prophylaxis Of Multiple Strains And Subtypes Of HIV-1”
36. Sambrook and Russell, Molecular Cloning: A Laboratory Manual, 3rd edition, Cold Spring Harbor Laboratory Press, Cold Spring Harbor, N.Y. 2001.
37. Tam, Proc. Natl. Acad. Sci. USA, 85:5409-5413 (1988)
38. D. Posnett et al., J. Biol. Chem., 263(4):1719-1725 (1988)
39. Tam, J., Vaccine Research and Developments, Vol. 1, ed. W. Koff and H. Six, pp. 51-87 (Marcel Deblau, Inc., New York 1992)
40. Buonaguro L et al, Virus-like particles as particulate vaccines, Curr. HIV Res., 2010 June, vol. 8(4): 299-309
41. Tabas, I. and Glass, C K, Anti-inflammatory Therapy in Chronic Disease: Challenges and Opportunities., Science, vol. 339:166 (January 2013)

What is claimed is:

1. An immunogenic composition comprising a peptide that induces in vivo a TNF monomer-specific antibody that binds an epitope of dissociated monomers of human TNF, the epitope being located wholly or partially within a TNF monomer-monomer interface region, wherein the binding disrupts assembly of the monomers into bioactive trimeric human sTNF.

2. The composition according to claim 1, further comprising an additional peptide that induces in vivo a TNF monomer-specific antibody that binds an additional epitope of dissociated monomers of human TNF, the epitope being located wholly or partially within a TNF monomer-monomer interface region, wherein the binding disrupts assembly of the monomers into bioactive trimeric human sTNF.

3. The composition according to claim 1, wherein the TNF monomer-specific epitope has the sequence PSDKPVAH, amino acids 8-15 of SEQ ID NO: 1 or PSDKPVAHV, amino acids 8-16 of SEQ ID NO: 1.

4. The composition according to claim 1, wherein the TNF monomer-specific epitope has the sequence EPIYLGGVF, amino acids 116 to 124 of SEQ ID NO: 1.

5. The composition according to claim 4, wherein the TNF monomer-specific antibody-inducing peptide is between 7 and 70 amino acids in length, comprising within that length the sequence of amino acids 8-15 or 8-16 of SEQ ID NO: 1.

6. The composition according to claim 1, wherein the TNF monomer-specific antibody-inducing peptide is between 7 and 70 amino acids in length, comprising within that length the sequence of amino acids 116-124 of SEQ ID NO: 1.

7. The composition according to claim 1, wherein a single immunogenic peptide induces antibody to TNF monomer-specific epitope A2 and epitope F.

8. The composition according to claim 1, further comprising in a linear or branched arrangement with the TNF monomer-specific antibody-inducing peptide or peptides, one or more components selected from a promiscuous T helper sequence, a linker sequence, a sequence of charged polar amino acids, and a lipopeptide cap selected from the group consisting of a di- or tri-palmitoyl-S-glyceryl-cysteine.

8. The composition according to claim 1, wherein said peptide or peptides are produced recombinantly or synthetically.

9. The composition according to claim 1, wherein said peptide or peptides is expressed within a recombinantly produced protein, optionally fused in frame with a carrier protein.

10. The composition according to claim 1, wherein said peptide or peptides is expressed in a virus-like particle.

11. The composition according to claim 1, which does not induce antibodies that bind transmembrane TNF (tmTNF) or affect the structure or bioactivity of tmTNF.

12. A method for treating or preventing a disease mediated by the activity of sTNF in a mammalian subject comprising: inducing in vivo in a mammalian subject in need thereof TNF monomer-specific antibodies that bind an epitope of dissociated monomers of human TNF, the epitope being located wholly or partially within an a TNF monomer-monomer interface region, wherein the binding disrupts assembly of the monomers into bioactive trimeric human sTNF.

13. A method for treating or preventing a disease mediated by the activity of sTNF in a mammalian subject comprising administering to a mammalian subject in need thereof an effective antibody-inducing amount of a composition of claim 1.

14. The method according to claim 13, wherein said disease is rheumatoid arthritis, ankylosing spondylitis, juvenile rheumatoid arthritis, psoriatic arthritis, psoriasis, obesity, metabolic syndrome, atherosclerosis, associated cardiovascular disease, Alzheimer's disease or a neurodegenerative disease.

15. The method according to claim 13, wherein the disease is a pathogenic effect of bioactive, trimeric sTNF produced during inflammation or during the course of an inflammatory disorder.

16. The method according to claim 13, which has no effect on the amount, concentration or bioactivity of tmTNF.

17. The method according to claim 13, wherein the subject's susceptibility to infection by an intracellular pathogen or to a malignancy is not increased by the administration.

18. The method according to claim 15, wherein the infection is tuberculosis, bacterial sepsis, invasive fungal infection, or histoplasmosis, or wherein the malignancy is lymphoma or hepatosplenic T-cell lymphoma.

19. A composition that induces in vivo in a mammalian subject monomer-specific antibodies that bind an epitope of dissociated monomers of human TNF, the epitope being located wholly or partially within an a TNF monomer-monomer interface region, for treating or preventing a disease mediated by the activity of sTNF in said subject, wherein the binding disrupts assembly of the monomers into bioactive trimeric human sTNF without affecting the amount, concentration or bioactivity of transmembrane TNF.
